(19) United States
(12) Patent Application Publication
(10) Pub. No.: US 2023/0149609 A1
(43) Pub. Date: May 18, 2023

(54) Holder for Lines of a Blood Treatment Apparatus

(71) Applicant: Fresenius Medical Care AG & Co. KGaA, Bad Homburg (DE)

(72) Inventor: Jenspeter Rau, Munchen (DE)

(21) Appl. No.: 17/919,358

(22) PCT Filed: Apr. 21, 2021
(86) PCT No.: PCT/EP2021/060349

§ 371 Date: Oct. 17, 2022

(30) Foreign Application Priority Data
Apr. 23, 2020 (DE) 102020111136.3

(51) Int. Cl. A61M 1/16; A61M 1/36

(52) U.S. Cl. CPC A61M 1/1652; A61M 1/3644; A61M 2209/082

(57) ABSTRACT

The present disclosure relates to a holder for holding a connector of a dialysis liquid inlet line and a connector of a dialysate outlet line, wherein the holder comprises an arm. The arm comprises a first retainer for receiving or holding the connector of the dialysis liquid inlet line and a second retainer for receiving or holding the connector of the dialysate outlet line. The present disclosure further relates to a blood treatment apparatus and to a method for setting up a blood treatment apparatus for a blood treatment session.

### CROSS-REFERENCE TO RELATED APPLICATIONS

The present application is the national stage entry of International Patent Application No. PCT/EP2021/060349, filed on Apr. 21, 2021, and claims priority to Application No. DE 10 2020 111 136.3, filed in the Federal Republic of Germany on Apr. 23, 2020, the disclosures of which are expressly incorporated herein in their entirety by reference thereto.

### TECHNICAL FIELD

The present disclosure relates to a holder, a blood treatment apparatus, and a method for preparing a blood treatment session. It also relates to a digital storage medium, a computer program product, and a computer program.

### BACKGROUND

Extracorporeal blood treatment is known from practice. Here, blood is taken from the patient and led extracorporeally along a blood circuit and for example through a blood filter. The blood filter comprises a blood chamber, through which blood is led, and a dialysis liquid chamber, through which dialysis liquid is led; said dialysis liquid being supplied to it via a dialysis liquid inlet line. These two chambers are separated from each other by a semi-permeable membrane. In practice, hollow fiber bundles with a semi-permeable fiber wall are usually used for this purpose. Thereby, the hollow fiber bundle is arranged inside a mostly cylindrical plastic housing. In this, during treatment, blood flows inside the fibers and the dialysis liquid flows along the outside of the fiber wall. The blood filter comprises at least one inlet and one outlet for the blood, at least one inlet for the dialysis liquid and one outlet for the dialysate.

Blood and dialysis liquid are usually passed through the blood filter in the countercurrent principle, wherein the blood is preferably passed through the blood chamber in an upward movement. The blood is purified in the blood filter and reinfused into the patient. The dialysis liquid is considered as used when it leaves the blood filter via a dialysate outlet line and, now being called effluent (also: effluat), is discarded.

### SUMMARY

An advantage of the present disclosure may be to provide an efficient and safe filling process.

For this purpose, the present disclosure proposes a suitable holder for the dialysis liquid inlet line and the dialysate outlet line of the blood treatment apparatus, as well as a further blood treatment apparatus. A method for preparing a blood treatment session is also to be provided. Furthermore, a digital storage medium, a computer program product and a computer program are to be specified.

Advantages of the present disclosure may be achieved by a holder as described herein and by a blood treatment apparatus as described herein. In addition, advantages may be achieved by a method as described herein, furthermore by a digital storage medium, a computer program product and a computer program as disclosed herein, respectively.

The present disclosure relates to a holder for use together with a blood treatment apparatus, in particular a dialysis machine.

The holder comprises an arm which in turn comprises a first retainer for receiving and/or holding, preferably releasably, a liquid inlet line, in particular a dialysis liquid inlet line, and a second retainer for, preferably releasably, receiving and/or holding a liquid outlet line, in particular a dialysate outlet line.

In this, the first retainer may be designed to receive and/or hold a connector of the liquid inlet line. Also the second retainer may be designed to receive and/or hold a connector of the liquid outlet line. Thus, a connector may be designed on both the liquid inlet line and on the liquid outlet line for connecting each of the aforementioned lines with a connector of a blood filter. These connectors may be inserted in the first or second retainer. In at least one embodiment, the connectors are releasably connected to the first or second retainer, respectively.

Similarly, in at least one embodiment, the first retainer may be designed and serve to receive and/or hold the liquid inlet line, e.g., being in contact with the material the line is made of, or, being in contact only with the line (in contrast to being in contact with the material the connector is made of, or in contrast to being in contact with the connector as well). In this case, the first retainer may be designed as a connector of the liquid inlet line. In other words, in this case the first retainer may fulfil the function of the connector, so that the first retainer may be connected to a first connector of a blood filter.

Likewise, in at least one embodiment, the second retainer may be designed to receive and/or hold directly the liquid outlet line. In this case, the second retainer may be designed as a connector for the liquid outlet line. In other words, in this case the second retainer may fulfil the function of the connector, so that the second retainer may be connected to a second connector of a blood filter. In this case, the first and second retainer are each a connector.

The term “holding” may mean that a component is held on the arm by a holding device, for example a tubular structure through which liquid can flow. “Holding” may also mean that the component through which the liquid can flow is formed as part of the arm and comprises two connecting sections for connecting to an inlet or for connecting to an outlet. In other words, the “retainer” may form an inseparable unit with the connector or a separable unit with the connector.

The present disclosure further relates to a blood treatment apparatus. It comprises a holder according to the present disclosure.

The present disclosure further relates to a method for setting up a blood treatment apparatus, in particular the blood treatment apparatus according to the present disclosure, prior to a blood treatment session or for performing a blood treatment session.

The method encompasses connecting a second connector of a blood filter to a connector associated with a dialysis fluid inlet line. It further encompasses connecting a first connector of the blood filter to a connector associated with a dialysate outlet line. The connection is made such that when liquid is supplied via the dialysis liquid inlet line, the supplied liquid flows against gravity within the blood filter towards the dialysate outlet line.

The method further encompasses filling the blood filter at least partially with liquid via the dialysis liquid inlet line.

The method further encompasses moving the connector assigned to the dialysate outlet line and the connector assigned to the dialysis liquid inlet line using a holder, in particular a holder according to the present disclosure, for holding or receiving both the dialysate outlet line and the dialysis liquid inlet line. The movement is preferably automatic, e.g., by provided devices or mechanisms.

The method further encompasses connecting the first connector of the blood filter to the connector associated with the dialysis liquid inlet line after the aforementioned connecting, and connecting the second connector of the blood filter to the connector associated with the dialysate outlet line, so that when fluid is supplied via the dialysis liquid inlet line, the liquid flows within the blood filter in the direction of gravity towards the dialysate outlet line.

In an alternative design of the method, which is also encompassed by the present disclosure, it comprises the following steps, which optionally include the step of setting up a blood treatment apparatus according to the present disclosure. As an alternative to this step, such a blood treatment apparatus may already be set up. In either case, it comprises a blood filter having a first connector for connecting the dialysis liquid chamber of the blood filter to a connector of a fluid line and having a second connector for connecting the dialysis liquid chamber of the blood filter to further connector of a further fluid line.

Furthermore, the blood treatment apparatus comprises a liquid inlet line, in particular a dialysis liquid inlet line, with a connector, a liquid outlet line, in particular a dialysate outlet line, with a connector, and a holder according to the present disclosure.

The connector of the liquid inlet line is received in the first retainer of the arm of the holder according to the present disclosure, connected to it or is designed as the first retainer, and the connector of the liquid outlet line is received in the second retainer of the arm of the holder, connected to it or is designed as the second retainer.

As a further step, the alternative method encompasses moving the first retainer and/or the second retainer together with the connectors received therein within and/or by the holder. In this, the first retainer itself may be formed as the connector or may have received the connector, and the second retainer itself may be formed as the connector or may have received the connector.

The movement is carried out such that at least one of the two retainers is in a new position after completion of the movement process, wherein the new position is different from the original position of the at least one retainer, wherein the retainer is moved or has been moved out of the original position preferably in a circular movement and/or along a circular path to assume the new position. Alternatively or in addition, the movement is optionally carried out such that after completion of this movement process the connector of the dialysis liquid inlet line is or can be assigned to the second connector of the blood filter, which during use of the blood filter is usually located further down the blood filter relative to the first connector of the blood filter, and that the connector of the dialysate outlet line is or can be assigned to the first connector of the blood filter, which during use is usually located further up the blood filter.

The alternative method further encompasses the step of moving the two retainers and the connectors received therein towards the blood filter using the holder and/or connecting the connector of the dialysis liquid inlet line to the second connector of the blood filter and connecting the connector of the dialysate outlet line to the first connector of the blood filter.

The alternative method further encompasses the step of filling, rinsing and/or priming the blood filter or sections thereof with dialysis liquid out of the dialysis liquid inlet line and into the blood filter.

As an optional further step, the alternative method encompasses the step of disconnecting the connector of the dialysis liquid inlet line from the second blood filter connector and disconnecting the connector of the dialysate outlet line from the first blood filter connector by moving the connectors of the two lines by moving the holder in the direction away from the blood filter.

An optional further step of the alternative method encompasses moving the first retainer and/or the second retainer together with the connectors received therein within and/or by the holder such that the two retainers are in their original position after completion of the movement process. Alternatively or in addition, the movement is optionally carried such that, after completion of the movement process, the connector of the dialysis liquid inlet line is assigned or can be assigned to the first connector of the blood filter and the connector of the dialysate outlet line is assigned or can be assigned to the second connector of the blood filter.

The alternative method further encompasses optionally the step of moving, by the holder, the connectors of the two lines towards the blood filter and/or connecting the connector of the dialysis liquid inlet line to the first connector of the blood filter and connecting the connector of the dialysate outlet line to the second connector of the blood filter.

Whenever a suitability or a method step is mentioned herein, the present disclosure also encompasses a corresponding programming or a configuration of a suitable device or of a section thereof.

A digital, in particular non-volatile, storage medium according to the present disclosure, in particular in the form of a machine readable carrier, in particular in the form of a floppy disk, memory card, CD, DVD, EPROM, FRAM (Ferroelectric RAM) or SSD (Solid State Drive), in particular with for example electronically or optically readable control signals, may interact with a blood treatment apparatus according to the present disclosure such that the steps of each of the methods according to the present disclosure are prompted and/or executed.

A computer program product according to the present disclosure comprises a program code volatile or saved on a machine-readable carrier or a signal wave for prompting the steps of any of the methods disclosed herein, when the computer program product interacts with a blood treatment apparatus according to the present disclosure.

A computer program product may, according to the present disclosure, be understood as, for example, a computer program which is stored on a carrier, an embedded system as a comprehensive system with a computer program (for example, an electronic device with a computer program), a network of computer-implemented computer programs (for example, a client-server system, a cloud computing system, etc.) or a computer on which a computer program is loaded, running, saved, executed or developed.

The term “machine-readable carrier” as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other memory or any other storage medium mentioned herein, respectively.

A computer program according to the present disclosure encompasses a program code by which the steps of any of the methods are executed by a blood treatment apparatus according to the present disclosure are effected or executed when the computer program runs on a computer.

Embodiments according to the present disclosure may comprise one, several or all of the following features in any combination, unless this is recognized as being technically impossible by the person skilled in the art. Advantageous developments of the present disclosure are each also subject-matter of the dependent claims.

In all of the following statements, the use of the expression “may be” or “may have” and so on, is to be understood synonymously with “preferably is” or “preferably has,” and so on respectively, and is intended to illustrate embodiments according to the present disclosure.

Whenever numerical words are mentioned herein, the person skilled in the art shall recognize or understand them as indications of numerical lower limits. Unless it leads the person skilled in the art to an evident contradiction, the person skilled in the art shall comprehend for example the specification of “one” as encompassing “at least one”. This understanding is also equally encompassed by the present disclosure as the interpretation that a numerical word, for example, “one” may alternatively mean “exactly one”, wherever this is evidently technically possible for the person skilled in the art. Both are encompassed by the present disclosure and apply to all numerical words used herein.

Whenever the expressions “top”, “bottom”, “left” or “right” are mentioned herein, the person skilled in the art understands the corresponding arrangement in the accompanying figures and/or during use. “Bottom” is closer to the center of the earth or to the lower edge of the figure than “top”.

Advantageous developments of the present disclosure are also subject-matter of embodiments as well as of figures and their description.

Whenever an embodiment is mentioned herein, it is then an exemplary embodiment according to the present disclosure.

Whenever an embodiment of the method according to the present disclosure is mentioned herein, then the statements made therein apply to the alternative method as well.

In some embodiments, the holder has a mechanism, e.g., a shifting mechanism, which is designed to interchange the position of the first retainer and the position of the second retainer.

In this, “interchangeable” may in some embodiments mean, in particular that the positions of the first retainer and the second retainer are interchangeable in space and/or relative to a further section of the holder or, in use, to a section of the blood treatment apparatus with which it is used. When the holder is used with, for example, a dialysis machine, the first retainer can thus be connected initially to the first connector of a blood filter and the second retainer can be connected initially to the second connector of the blood filter. By moving the first and second retainer using the mechanics of the holder, the position of the first retainer can now be swapped with the position of the second retainer, i.e., the two retainers exchange place or position so that the first retainer is connected to the second connector of the blood filter and the second retainer is connected or can be connected to the first connector of the blood filter.

In some embodiments, the first retainer and/or the second retainer are provided on the arm at an invariable distance from each other, i.e., with an invariable distance between each other or from each other.

In some embodiments, the first retainer and/or the second retainer is arranged to be relatively movable such that a fluidic connection, in particular an immediate or direct fluidic connection, can be or is established between the liquid inlet line held in the first retainer and the liquid outlet line held in the second retainer, in other words, a short circuit may therefore be established between the liquid inlet line and the liquid outlet line, in particular without an intermediate piece or in particular only with components of the holder. A direct, fluidic connection may thus be a connection between the liquid inlet line and the liquid outlet line, which does not take place via the dialyzer or blood filter.

For example, the short-circuit connection may be designable such that the blood treatment apparatus may have a line section with the ends of which the connectors on the first retainer and the second retainer are connectable. In other words, instead of the blood filter, the retainers are connected or may be connected to a transition tube or transition hose in the form of a short circuit. This connection with the transition tube/hose may be made when the retainers and thus also the connectors are positioned differently than when they are connected to the blood filter. For this purpose, the arm may be brought into a position that differs from the position in which the blood filter may be connected.

In a further embodiment, the transition tube or the transition hose may be movably arranged and, for the connection to the connectors on the first and second retainers, displaceable into a corresponding position, for example moved into or onto the connectors.

The first retainer and/or the second retainer may thus be provided such that they may be pivotable towards each other, for example by at least 30 degrees, 45 degrees, but preferably substantially 90 degrees, so that the opening of the liquid outlet line and the opening of the liquid inlet line lie opposite to each other. In addition, a fluid line may be formed in the holder, e.g., inside the arm, on the arm or adjacent to it. In this way, a direct or immediate fluidic connection between the liquid inlet line and the liquid outlet line may be formed by pivoting the first and second retainer, preferably by 90 degrees, and thus connecting the liquid inlet line and the liquid outlet line to the fluid line formed in the arm.

In this, at least one, alternatively two, fluid line(s) may be formed within the arm. Via a port assigned to each of the first retainer and the second retainer, which allows the folded-in liquid inlet line or liquid outlet line to be connected to the fluid line formed in the arm, the liquid may flow through the fluid line(s) to and from the first and/or the second retainer. This port of the first or second retainer may also be associated with valves that may control the flow of fluid through the fluid line(s) in the arm.

In some embodiments, the first retainer and/or the second retainer are arranged so as to be movable or displaceable along the arm by a guide, in particular a rail, a slide or slide guide or the like. The two retainers may thus move towards each other and/or further apart. The two retainers may interchange their positions on the arm by the displacement or movement.

The shifting mechanism may be identical to the rail or guide disclosed herein in some embodiments. However, the shifting mechanism on the one hand and the rail or guide on the other hand may also be different devices in other embodiments.

In some embodiments, the holder according to the present disclosure comprises an actuator for rotating the arm and/or for displacing or translating the first retainer and/or the second retainer along the arm. The actuator may comprise a drive motor. It may comprise a chain, an endless guide or the like. It may be controlled electrically. The actuator may be a pneumatic or hydraulic drive. Alternatively, the actuator may be an electric motor. In this, the actuator may be mounted close to or at or in the blood treatment apparatus and the drive shaft may drive a toothed gearwheel that drives a toothed belt or chain. This allows, for example, the first and second retainer to be movable along the arm. The arm may also be mounted directly or immediately on the drive shaft or on the rotation section for rotating the arm discussed below. In addition, a second actuator may be provided for rotating and/or shifting or translating. Alternatively, only one actuator may be provided for either twisting and shifting or twisting.

The actuator discussed above may be the traversing mechanism or a part of it. It may be provided in addition to the traversing mechanism.

In some embodiments, the arm is rotatable about or related to a section of the holder, preferably about 180 degrees or substantially about 180 degrees.

In some embodiments the holder comprises a connecting section. This is designed for its preferably releasable connection to a rotatably arranged rotation section of a blood treatment apparatus. When it is connected, a rotation-proof connection is created between the connecting section and the rotation section. If the connecting section is rotated by the rotation section, the holder, or at least the arm, rotates about an axis of rotation. The latter may be perpendicular to a longitudinal axis of the arm.

In several embodiments, the arm is designed to be variable in length, in particular foldable or telescopic. A device or a corresponding, in particular motor-driven, actuator for varying the length of the arm may be provided on the holder or on the blood treatment apparatus, as may a device or mechanism for, in particular motor-driven, folding or pivoting at least a section of the arm about a folding axis and relative to another section of the arm. Using this device and/or mechanism, the first retainer may be pivoted towards and/or pivoted away from the second retainer and/or vice versa.

As in the embodiment described above, the length-variable arm may also be arranged in a rotatable manner. This combination of length-variable and rotatable arm may have the advantage that the space required for the rotational movement can be kept small. For this purpose, in order to exchange the positions of the retainers, it may optionally be possible to reduce the length of the arm in a first step, to rotate the arm with the retainers in a second step, and to increase the length of the arm to the previous length in a third step.

In addition, a short circuit between the liquid inlet line and the liquid outlet line may be achieved by moving the arm.

In particular, in the case of a foldable arm, the first retainer may approach the second retainer. If the liquid inlet line and liquid outlet line are received in the retainers, these may be plugged into one another or otherwise connected to each other by folding the arm. In this way a fluidic connection between these two lines may be formed, in particular an immediate or direct connection, or by a connector which may be part of the holder. Alternatively or additionally, the first retainer and/or the second retainer may be folded into the arm to achieve a short circuit as described above. Further alternatively or additionally, the arm may assume a position in which there is no connection to the blood filter. Here, for example, by moving the first and second retainer on the arm and/or by rotating the arm by an angle of 90 degrees or less, a short-circuit tube may be approached, which is located for example on or in the blood treatment apparatus.

In some embodiments, a connector of a dialysis liquid inlet line is received in the first retainer and a connector of a dialysate outlet line is received in the second retainer.

In some embodiments, the blood treatment apparatus comprises a dialysis liquid inlet line with a connector and a dialysate outlet line with a connector.

In several embodiments of the blood treatment apparatus, the connector of the dialysis liquid inlet line is received or held in the first retainer, for example manually releasable, releasable only by using a tool or non-releasably, and the connector of the dialysate outlet line is received or held in the second retainer, for example manually releasable, releasable only by using a tool or non-releasably. In another embodiment, the connector is designed integrally with the first retainer and/or with the second retainer.

In some embodiments the blood treatment apparatus comprises an actuator, a programming and/or a mechanism for placing a cover on the first connector, in particular on its free end, and/or for removing it from the first connector, alternatively or in addition to placing and/or removing a cover on or from further connectors, for example on or from the second connector.

In several embodiments of the holder or of the blood treatment apparatus, the connector of the dialysis liquid inlet line and/or the connector of the dialysate outlet line comprises a cover, herein disclosed, which is detachably placed on or inserted in it.

In some embodiments, the connector of the dialysis liquid inlet line, the connector of the dialysate outlet line and/or the cover, or a section thereof, respectively, has a tapering, in particular conical, shape or the shape of a (hollow) truncated cone.

In several embodiments of the blood treatment apparatus according to the present disclosure, it comprises a blood filter (also: dialyzer) or is connected to such a filter. The latter in turn comprises a first and a second connector which serve to connect it, in particular to connect its dialysis liquid chamber, to the connector of the dialysis liquid inlet line and to the connector of the dialysate outlet line, respectively.

In some embodiments of the blood treatment apparatus, the first and/or the second connector of the blood filter, the connector of the dialysis liquid inlet line and/or the connector of the dialysate outlet line comprise a drip protection, in particular a septum, for example made of silicone.

The drip protection may be arranged at the end of the lumen of the respective connector and may comprise a flexible or elastic membrane or disk covering the lumen of the connector. This membrane or disc may comprise at least one or more slots.

Alternatively or in addition, the drip protection may comprise a valve that opens only at or above a certain liquid pressure. When supplying the liquid, e.g., during filling of the blood filter, it may be possible to build up a pressure that allows the supplied liquid to overcome the drip protection. When the supply is stopped, the pressure is reduced and, when the connectors are subsequently disconnected from the blood filter connectors, no or almost no liquid escapes via the connectors due to the drip protection.

Alternatively or additionally, when connecting to the blood filter, an actuator inside the lumen of the blood filter may penetrate the membrane or the disk or open the valve. When disconnecting, this actuator is pulled out again and the drip protection closes the connector again.

When using a membrane or a disk as drip protection or as part of it, the actuator itself may be tubular and the fluidic connection to the blood filter is established through the inner lumen of the tube. When using a valve, the actuator may mechanically change the position of the closure element of the valve and thereby release the fluidic connection.

In several embodiments of the blood treatment apparatus, the blood treatment apparatus comprises a holding device or stock for holding or receiving several covers, for example, in a stacked or nested manner.

In some embodiments, the blood treatment apparatus comprises a device for changing the length of the arm if the arm is designed to be variable in length. Alternatively or additionally, it has a device for displacing or moving the first retainer and/or the second retainer along the arm.

In several embodiments of the blood treatment apparatus, it comprises a rotation section and a drive unit therefor. The rotation section is rotatably mounted and may be controlled in order to rotate about an axis of rotation. It also serves to connect it to the connecting section of the holder, producing a rotation-proof connection between the rotation section and the connecting section.

In some embodiments of the blood treatment apparatus according to the present disclosure, the blood treatment apparatus comprises a first connector on the machine-side and a second connector on the machine-side which are fluidically interconnected, for which reason fluid introduced into the first of these connectors would (preferably only inevitably) exit the second connector, and vice versa. These two connectors are provided to the blood treatment apparatus (usually at a housing or in an outer wall thereof) and are arranged at a structurally predetermined distance from each other.

In some embodiments, the arm is variable in length or otherwise variable such that its first and second retainers may be brought to a distance from each other so that the two connectors received in them may be inserted, plugged or otherwise connected to the first or second connector on the machine-side, respectively, automatically or by a suitable mechanisms and/or controls of the relevant device. This creates a short circuit for the dialysis liquid, allowing it to bypass the dialysis liquid chamber of the blood filter.

In some embodiments, the blood treatment apparatus is designed as a hemodialysis apparatus, a hemofiltration apparatus or as a hemodiafiltration apparatus, in particular as an apparatus for the acute, for the chronic renal replacement therapy or for the continuous renal replacement therapy (CRRT).

In several embodiments of the method according to the present disclosure, the movement of the first retainer and/or of the second retainer together with the connectors received therein within and/or by the holder is or comprises a folding or pivoting movement by which the two retainers or the connectors received therein are moved towards each other. By folding the connector of the dialysis liquid inlet line and the connector of the dialysate outlet line towards each other (and then connecting them), it is possible to short-circuit both lines, allowing the dialysis liquid to bypass the dialysis liquid chamber of the blood filter. This arrangement may be denoted as “bypass”.

In several embodiments, the method further comprises moving the connector of the dialysis liquid inlet line and/or the connector of the dialysate outlet line towards each other within and/or by the holder, so that a fluidic connection between the dialysis liquid inlet line and the dialysate outlet line is formed.

Additional devices, such as adapters for the connectors and/or hose line sections, which are also covered by the present disclosure, may be necessary for connecting.

In some embodiments, the connector, its lumen, and/or a flexible tube material of the dialysate liquid inlet line and/or the dialysate outlet line do not extend or protrude beyond the first receptacle of the arm or the second receptacle of the arm, respectively. In several embodiments, the dialysis liquid inlet line and/or the dialysate outlet line terminate or end where they, or their respective connector, are accordingly received with the first receptacle or the second receptacle, respectively. A method according to the present disclosure may for this purpose be provided and prepared accordingly.

In some embodiments, the first receptacle of the arm and the second receptacle of the arm are not intended and/or embodied to guide, receive, or hold together both the dialysis liquid inlet line and the dialysate outlet line.

In several embodiments, the first receptacle of the arm and the second receptacle of the arm are not intended and/or embodied to guide, receive, and/or hold together both the dialysis liquid inlet line and the dialysate outlet line.

In some embodiments, the first receptacle of the arm and the second receptacle of the arm are provided and embodied to determine and/or fix the distance between the connector of the dialysis liquid inlet line being inserted as intended and/or the connector of the dialysis outlet line being inserted as intended. In this, a design may be provided by which the distance between the first receptacle of the arm and the second receptacle of the arm may only change by the same distance as the distance between the connector of the dialysis liquid inlet line being inserted as intended and the connector of the dialysis outlet line being inserted as intended.

In some embodiments, the arrangement of the first receptacle of the arm and the second receptacle of the arm in the space or area determines the position of the connector of the dialysis liquid inlet line being inserted as intended and the connector of the dialysis outlet line being inserted as intended in the space.

In several embodiments, the arm is not connected directly or indirectly to a current conductor and/or is not restraint-guided to the latter.

In some embodiments, the movement of the arm is not coupled to the movement of a current conductor.

In several embodiments, the first receptacle of the arm and the second receptacle of the arm are provided or arranged on the blood treatment apparatus to be movable, particularly for a rotation, relative to a housing of the blood treatment apparatus or a mounting surface of the blood treatment apparatus.

In several embodiments, the arm and/or the first receptacle and/or the second receptacle of the arm is formed completely or partially within a housing of the blood treatment apparatus.

In some embodiments, the first receptacle of the arm, the second receptacle of the arm, and/or the arm itself is not suitable and/or provided to hold, be connected to, and/or comprises no engagement nor connection device for the blood filter or dialyzer.

Some or all embodiments according to the present disclosure may comprise one, several or all of the advantages mentioned above or in the following.

During a blood treatment session, the blood chamber is preferably perfused from bottom to top. Based thereon, the dialysis liquid chamber of the blood filter is preferably perfused from top to bottom with dialysis liquid in order to purify the blood to be treated there via the semi-permeable membrane following the countercurrent principle with the blood chamber of the blood filter. Since the blood filter is usually filled with air at the beginning of the treatment, depending on the type of blood filter used, a so-called priming process may be provided, which removes the air also from the dialysis liquid chamber of the blood filter and concurrently fills it with dialysis liquid. For this purpose, the dialysis liquid perfuses the blood filter from bottom to top, i.e., against gravity, in order to ensure a complete degassing of the dialysis liquid chamber. This is advantageously possible by the present disclosure without human intervention, which may help to save time and work.

In other words, before treatment, the blood filter in both chambers must be filled with physiologically safe liquid, i.e., by this filling liquid, the air in the blood filter must be displaced by liquid. In this, it is important that this air is removed as completely as possible also from the dialysis liquid chamber, because air remaining on the outside of the fiber wall means that this area of the wall cannot contribute to blood purification which would therefore reduce the efficiency of the treatment. This filling is particularly efficient when filling is done from below, i.e., against gravity. With the present disclosure, this efficient filling may be automated. Thereby, according to the present disclosure, it is also not necessary for the blood filter to be rotatable. The greatest possible flexibility of the design is thus achieved by the present disclosure.

By using the present disclosure, it may further be prevented that, for example, the staff would have to manually turn or reverse the blood filter with the aforementioned objective and then have to return it to its treatment position.

Since the present disclosure also allows the manual insertion or re-positioning or re-insertion of connectors, for example for priming, starting the blood treatment session and/or creating a bypass for the dialysis liquid without human intervention, time and work may be saved here.

The use of the covers disclosed herein may help to reduce the risk of contamination during the preparation of the blood treatment session and thus indirectly increase patient safety.

All advantages achievable with the method according to the present disclosure may be achieved undiminished also with the devices according to the present disclosure, and vice versa.

### BRIEF DESCRIPTION OF THE DRAWINGS

In the following, the present invention is purely exemplarily described with reference to the accompanying figures, in which the same reference numerals denote similar or identical components. The following applies:

FIG. 1 shows a highly simplified representation of a blood treatment apparatus, connected to an extracorporeal blood circuit and to a holder;

FIG. 2 shows a simplified representation of a holder in a first embodiment;

FIG. 3 shows a simplified representation of the holder shown in. FIG. 2 with inserted connectors to the left of a blood filter of a blood treatment apparatus;

FIG. 3a shows a simplified representation of the holder, shown in FIG. 3, with a collapsed arm during the method described herein;

FIG. 4 shows a simplified representation of a holder in a second embodiment, to the left of a blood filter of a blood treatment apparatus;

FIG. 5a shows the holder shown in FIG. 4, (with a view from the right relative to the illustration in FIG. 4) at the beginning of an embodiment of the method described herein;

FIG. 5b shows the holder shown in FIG. 5a, during a method as described herein;

FIG. 5c shows the holder shown in FIG. 5a, during a method as described herein;

FIG. 6 shows a simplified representation of an exemplary course of a method according to the present disclosure;

FIG. 7a shows a simplified representation of the holder in a further embodiment;

FIG. 7b shows a simplified representation of the holder shown in FIG. 7a, in a folded position;

FIG. 8a shows a design of a cover before its use in an exemplary storage position;

FIG. 8b shows the use of the cover, shown in FIG. 8a;

FIG. 8c shows a further design of a cover and its use; and

FIG. 8d shows the design of the cover, shown in FIG. 8c, after disconnecting the connectors.

### DETAILED DESCRIPTION

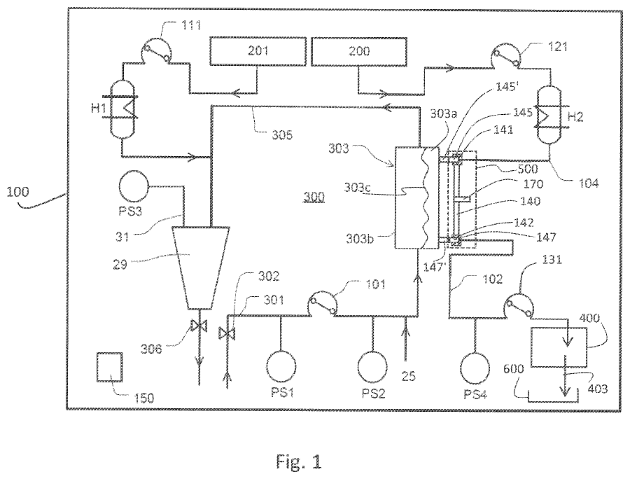

FIG. 1 shows a highly simplified representation of a blood treatment apparatus 100 according to the present disclosure, optionally connected to an extracorporeal blood circuit 300, to an effluent bag 400 and to a holder 500 according to the present disclosure during a blood treatment session.

The extracorporeal blood circuit 300 comprises a first line 301, here an arterial line section.

The first line 301 is in fluid communication with a blood treatment device, here exemplarily a blood filter or dialyzer 303 having a first connector 145′ and a second connector 147′. The blood filter 303 comprises a dialysis liquid chamber 303a and a blood chamber 303b which are separated from each other by a mostly semi-permeable membrane 303c.

The extracorporeal blood circuit 300 further comprises at least a second line 305, here a venous line section. Both the first line 301 as well as the second line 305 may serve as their connection to the vascular system of the patient, not shown here.

The first line 301 is optionally connected with a (first) hose clamp 302 for blocking or closing the line 301. The second line 305 is optionally connected to a (second) hose clamp 306 for blocking or closing the line 305.

The blood treatment apparatus 100 which is represented in FIG. 1 only by some of its devices and merely schematically, comprises a blood pump 101. During the patient's treatment, the blood pump 101 conveys blood through sections of the extracorporeal blood circuit 300 and towards the blood filter or dialyzer 303. This is illustrated by the small arrows, which generally indicate the flow direction in each of the figures.

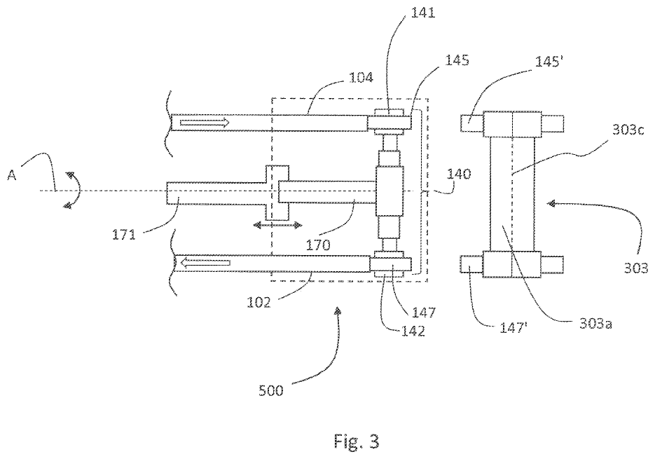

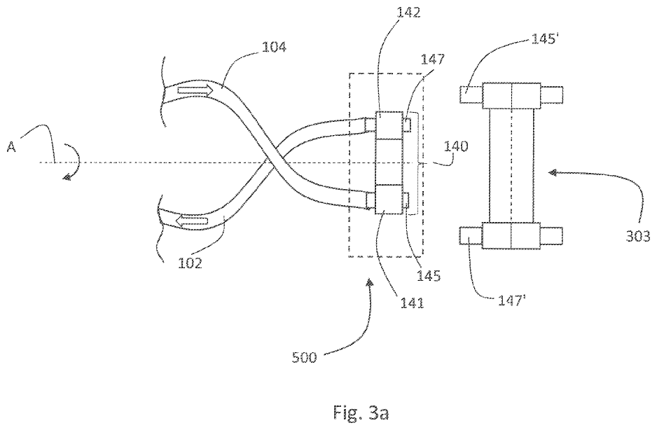

Using a pump for dialysis liquid 121, which may be designed as a roller pump, as an otherwise occluding pump or as a membrane pump or impeller pump, fresh dialysis liquid is pumped, e.g., from a source 200 along the dialysis liquid inlet line 104 via its connector 145 and via the first connector 145′ of the blood filter 303 into the dialysis liquid chamber 303a. The dialysis liquid leaves the dialysis liquid chamber 303a via the second connector 147′ of the blood filter 303 and the connector 147 of the dialysate outlet line 102, connected thereto, towards the optional effluent bag 400, or towards a basin and is herein referred to as effluent. The connector 145 of the dialysis liquid inlet line 104 is received or held in the first retainer 141 at an arm 140 of the holder 500 according to the present disclosure. The connector 147 of the dialysate outlet line 102 is received or held in the second retainer 142 at the arm 140 of the holder 500. This is described in more detail regarding FIG. 3.

The effluent may be discarded out of the optional bag 400 into the basin 600 using the effluent outlet line 403. In some embodiments (not shown here), the effluent is directly discarded into a basin 600 by the dialysate outlet line 102.

The source 200 may, for example, be a bag or a container. The source 200 may also be a fluid line through which on-line and/or continuously generated or mixed liquid is provided, for example a hydraulic outlet or hydraulic connection port of the blood treatment apparatus 100.

A further source 201 with substituate may be optionally provided. It may correspond to the source 200 or be a separate source.

A control device or closed-loop control device 150 being only roughly outlined may be configured to control or regulate the blood treatment apparatus 100. It may be in cable or wireless signal communication to any of the components of the medical set, especially or in particular to the blood pump 101, for controlling or closed-loop controlling the blood treatment apparatus 100.

In addition to the aforementioned blood pump 101, the arrangement shown in FIG. 1 further comprises purely optionally a series of further pumps, in each case optional, namely the pump 111 for substituate, the pump 121 for dialysis liquid and the pump 131 for the effluent.

The pump 121 is provided in order to supply the blood filter 303, using a dialysis liquid inlet line 104, with dialysis liquid from a source 200, for example out of a bag, via an optionally present bag heater H2 with a heating bag.

The dialysis liquid, thus supplied, exits again from the blood filter 303 via a dialysate outlet line 102 supported by the pump 131, and may be discarded, as described herein.

Upstream of the blood pump 101, an optional arterial sensor PS1 is provided. During a patient's treatment it measures the pressure in the arterial line.

Downstream of the blood pump 101, but upstream of the blood filter 303 and, if provided, upstream of an addition site 25 for an anticoagulant, for example Heparin, a further optional pressure sensor PS2 is provided. It measures the pressure upstream of the blood filter 303 (“pre-hemofilter”). The addition site for the anticoagulant may alternatively be arranged upstream of the blood pump 101.

Again, a further pressure sensor to measure the filtrate pressure of the blood filter 303 may be provided as PS4 downstream of the blood filter 303, however preferably upstream of the pump 131 in the dialysate outlet line 102.

Blood, which leaves the blood filter 303, passes through an optional venous blood chamber 29, which may comprise a de-aeration device 31 and/or a further pressure sensor PS3.

The optional pump 111 is provided to supply the second line 305 with substituate from the optional source 201, for example out of a bag, and via an optionally present bag heater H1 with a heating bag.

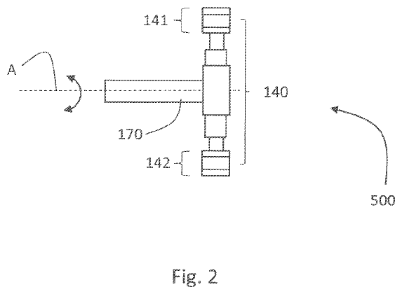

FIG. 2 shows a simplified representation of a holder 500 according to the present disclosure in a first embodiment. The holder 500 comprises the arm 140, which in turn comprises the first retainer 141, into which the connector 145 of the dialysis liquid inlet line 104 (see FIG. 3), is inserted or received, preferably releasably, or by which the connector 145 may be held.

Furthermore, the arm 140 comprises the second retainer 142, in which the connector 147 of the dialysate outlet line 102 (see FIG. 3) is inserted or received, preferably releasably, or by which the connector 147 may be held.

The connectors 145 and/or 147 may, as stated above, be part of the dialysis liquid inlet line 104 or the dialysate outlet line 102. Alternatively, they can be part of the holder 500.

In this embodiment, the first retainer 141 and the second retainer 142 are provided at a variable distance from each other, opposite to each other, each at the end of the arm 140.

According to the present disclosure, the position of the first retainer 141 and the position of the second retainer 142 may be interchanged. With or due to such an interchange of the positions, the position of the connectors 145, 147 may also be interchanged.

In the example of FIG. 2, the arm 140 is designed to be variable in length in order to change or adjust the distance. In particular, it is telescopic, with optional three sections (e.g., shaped as sleeves) that are slidable into each other, at least in sections. By pushing the individual sections of the arm 140 into each other, the distance between the first retainer 141 and the second retainer 142 may be reduced, this is shown and explained in more detail regarding FIG. 3a.

An optional connecting section 170 serves to connect the holder 500 to, e.g., a rotation section 171 of the blood treatment apparatus 100, exemplarily shown in FIG. 3, advantageously by creating a rotation-proof connection. Connecting may encompass plugging in and optionally furthermore latching, interlocking or the like.

Turning or rotating the holder 500 by the connecting section 170, when the original distance between the first retainer 141 and the second retainer 142 is re-established or restored, finally causes the positions of the first retainer 141 and the second retainer 142 to be interchanged.

FIG. 3 shows a simplified representation of the holder 500 according to the present disclosure, shown in FIG. 2, with the connector 145 of the dialysis liquid inlet line 104 inserted in the first retainer 141 and the connector 147 of the dialysate outlet line 102 inserted in the second retainer 142. The holder 500 is arranged on the left of a blood filter 303 of a blood treatment apparatus 100 according to the present disclosure, not shown further in FIG. 3.

The blood filter 303 comprises the first connector 145′ and the second connector 147′. They serve to fluidically connect the interior of the dialysis liquid chamber 303a of the blood filter 303 with the interior of the dialysis liquid inlet line 104 or of the dialysate outlet line 102.

In this embodiment, the first retainer 141 and the second retainer 142 are each arranged at the opposite ends of the arm 140.

The optional connecting section 170 serves to connect the holder 500 to a rotation section 171 of the blood treatment apparatus 100, preferably in a rotation-proof manner. The rotation of the rotation section 171, and thus indirectly also of the arm 140, about a rotation axis A may take place clockwise or counterclockwise. The rotation by 180° may also be done clockwise or counterclockwise alternately. In this way it may be prevented that the inlet line 104 and the outlet line 102 are twisted together to an impermissible extent.

In the example of FIG. 3, the arm 140 is optionally variable in length, here telescopic. This function is described in more detail regarding FIG. 3a.

The flow direction of the fluid conveyed in lines 102, 104, which is always advantageously unchanged or constant (i.e., during priming, rinsing as well as during subsequent treatment) is indicated in FIG. 3 and in the other figures by an arrow within the respective lumen.

Actuators and other devices may be provided for shifting the connecting section 170 or connecting the connectors 145, 145′, 147, 147′ in pairs (in FIG. 3 a left-right movement) and for collapsing and extending the arm 140 (in FIG. 3 an up-down movement). These are not shown in FIG. 3 nor in the other figures. The drive of the arm 140 and/or of the holder 500 and/or the of relative movement between connecting section 170 and rotation section 171 may optionally be done by an electric motor, pneumatically or hydraulically.

In the area of their interfaces with the connectors 145′, 147′ of the blood filter 303, the connectors 145, 147 may have drip protection, for example septa or valves which seal the inner lumens of connectors 145, 147 through which the fluid is passed and prevent liquid leakage during or after disconnection.

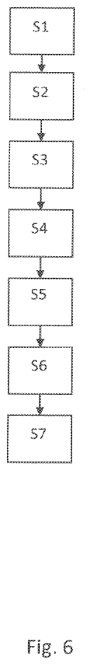

FIG. 3a shows a simplified representation of the holder 500 according to the present disclosure, shown in FIG. 3, while step S2 of an embodiment of the method described regarding FIG. 6 is carried out as an example of a method according to the present disclosure.

For reasons of clarity, the connecting section 170 and the rotation section 171 (see FIG. 3) are not shown again in FIG. 3a.

The holder 500 comprises the optionally length-variable arm 140 with optional three, sections (here shaped as sleeves) that are slidable into each other, at least in sections. FIG. 3a shows the arm 140 after it has been pushed together, therefore only its outermost sleeve is visible. As a result of the arm 140 being pushed together, the distance between the first retainer 141 and the second retainer 142 has noticeably or clearly decreased. The distance between the connectors 145 and 147 inserted in the retainers 141 and 142 has thus also indirectly decreased.

The arm 140 shortened in this way may, therefore, even if space is limited, be rotated clockwise, preferably by an angle of 180°, about its axis of rotation A which is perpendicular to its longitudinal axis. Due to the rotation that has already taken place by 180° relative to the position of the arm 140 in FIG. 3, the rear sides of the first retainer 141 and the second retainer 142 are visible in FIG. 3a.

The arm 140 may now be extended to its full length again in the further course of step S2 of the method according to the present disclosure, shown in FIG. 6.

Subsequently, for example in the course of a rinsing or priming process, in step S3 of the method shown in FIG. 6, on the one hand, connector 145 of the dialysis liquid inlet line 104 may be connected to the second connector 147′ of the blood filter 303 arranged comparatively low or below, and on the other hand, connector 147 of the dialysate outlet line 102 may be connected to the first connector 145′ of the blood filter 303, arranged comparatively high or above (not shown in FIG. 3a). The blood filter 303 may now be filled with liquid from the dialysis liquid inlet line 104 from bottom to top, which may advantageously contribute to optimal degassing of the blood filter 303.

After the rinsing or filling or priming process and after disconnecting the connectors 145, 147 of lines 102, 104 from connectors 145′, 147′ of the blood filter 303 (here, for example, by moving the connecting section 170 of the holder 500 to the left, i.e., by pulling back the holder 500), the arm 140 may be shortened again as described above in order to, despite small or little available space, rotate it back into the position shown in FIG. 3, which preferably is done in the opposite direction to the direction of rotation mentioned first, in order to prevent further twisting of the dialysis liquid inlet line 104 with the dialysate outlet line 102 and their damage caused by said twisting.

In order to effect or prompt that the dialysis liquid may flow through the blood filter 303 in the desired direction during the blood treatment, in the example of FIG. 3 from top to bottom, the arm 140, which has been returned back in the meantime, is extended to its original full length as shown in FIG. 3. Furthermore, on the one hand, connector 145 of the dialysis liquid inlet line 104 is connected to the first connector 145′ of the blood filter 303 and, on the other hand, connector 147 of the dialysate outlet line 102 is connected to the second connector 147′ of the blood filter 303, which is also no longer shown in FIG. 3a. This may be done, for example, by shifting the connecting section 170 again, this time to the right.

As stated, the length variability of the arm serves to accommodate to less place availability. Therefore, the same holder may also be used without a length-variable arm. The length variability of the arm is, therefore, only an optional, not an essential feature of the arm.

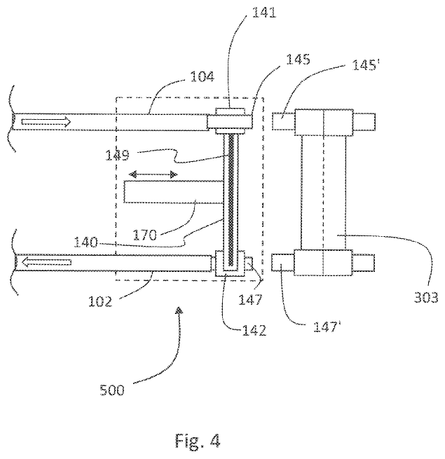

FIG. 4 shows a simplified representation of the holder 500 according to the present disclosure with inserted connectors 145, 147 to the left of the blood filter 303 of the blood treatment apparatus 100 according to the present disclosure in a second embodiment.

The arrangement of FIG. 4 largely corresponds to the arrangement of FIG. 3, to which reference is made in order to avoid repetition.

The positions of the first retainer 141 and the second retainer 142 shown in FIG. 4 may be referred to as their initial positions.

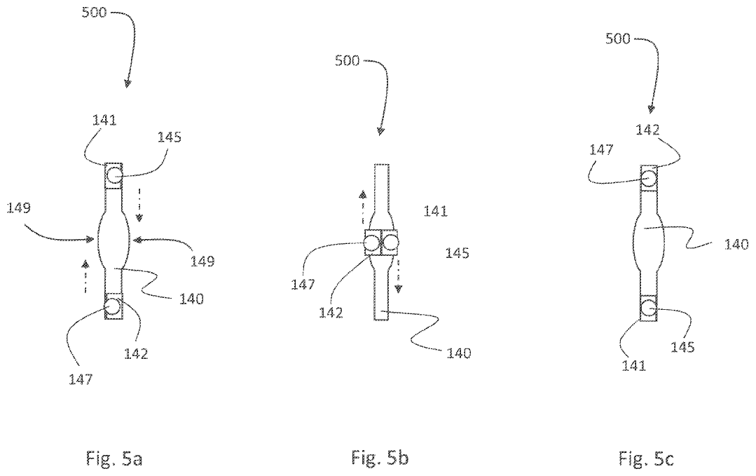

In this embodiment, the first retainer 141 and the second retainer 142 are provided to be movable along the arm 140, which in this embodiment is optionally not variable in length itself. To move the retainers 141, 142 along the arm 140, optionally rails 149 may be provided on the arm 140, for example respectively one rail on both sides (i.e., front and rear in FIG. 4). The function of these rails 149 is described in the following and in FIG. 5a to FIG. 5c in more detail.

In the embodiment of FIG. 4, the first retainer 141 may be moved in the rail 149 along the arm 140 and on its front side facing the viewer towards the bottom (towards the second retainer 142) and thus shifted or displaced. Likewise, the second retainer 142 may be moved in a rail (not visible in the representation in FIG. 4) along the arm 140 and on its rear side, which is behind the drawing plane, upwards (towards the first retainer 141) and thus shifted.

The arm 140 may be designed such that the two retainers 141 and 142 may be moved past each other, e.g., in the middle of the arm 140.

For this purpose, as described in more detail regarding an embodiment of FIGS. 5a to 5c, the arm 140 may have two rails, or linear guides, on its front side and rear side as described herein. The retainers may be moved in them, for example by a chain drive or a driven toothed belt. Alternatively, a rail may be designed on only one side of the arm, which rail branches in a central section for a partial section and then merges together again. Thus, the retainers moving towards each other may move past each other in the middle section through the rails, here, extending parallel.

After or as soon as the first retainer 141 has been moved completely or sufficiently far down and the second retainer 142 has been moved completely or sufficiently far up, the connector 145 of the dialysis liquid inlet line 104 received in the first retainer 141 may be connected to the second connector 147′ of the blood filter 303, and the connector 147 of the dialysate outlet line 102 received in the second retainer 142 may be connected to the first connector 145′ of the blood filter 303, since these connector pairs are now opposite to each other or facing each other, this connecting may take place for example by moving the connecting section 170 of the holder 500 to the right.

Moving the retainers 141 and 142 may be pushing, pulling, sliding and/or shifting.

For the sake of clarity, only the arm 140 of the holder 500 according to the present disclosure with the first retainer 141 and the second retainer 142 is shown in FIGS. 5a to FIG. 5c. The connector 145 of the dialysis liquid inlet line 104 is received in the first retainer 141, and the connector 147 of the dialysate outlet line 102 is received in the second retainer 142. The further reference numerals refer to the description of FIG. 1 to FIG. 4.

FIG. 5a shows the holder 500 according to the present disclosure in the design of FIG. 4 with a view from the right (with regard to the illustration of FIG. 4) at the beginning of the method according to the present disclosure in an embodiment of the method.

The first retainer 141 is positioned at the upper end of the arm 140, the second retainer 142 at its lower end. This arrangement in turn represents the initial positions of the retainers 141 or 142 of the holder 500.

On the left and right in or on the arm 140, for example one rail 149 or guide may be sunk. This is not visible in FIG. 5a. Alternatively or additionally, other mechanisms for moving the retainers 141 and 142 may also be provided.

In the context of an embodiment of the method according to the present disclosure, the retainers 141, 142 are each individually moved in the direction in which the corresponding dash-dotted arrow points, respectively.

FIG. 5b shows the holder according to the present disclosure, shown in FIG. 5a, during a step of the method according to the present disclosure, which follows the position shown in FIG. 5a.

The two retainers 141 and 142 were each moved individually to the middle of the arm 140 (corresponding to step S2 in FIG. 6). The design of the arm 140 of the holder 500 offers sufficient space in this embodiment so that the first retainer 141 and the second retainer 142 may be moved past each other.

In some embodiments, such positioning of the first retainer 141 and of the second retainer 142 in the middle of the arm 140, along with other possible designs of a short circuit and their temporary remaining there, may be used to allow the dialysis liquid or another fluid in the dialysis liquid inlet line 104 to bypass the dialysis fluid chamber 303a of the blood filter 303. This may be done, for example, by connecting connector 145 of the dialysis liquid inlet line 104 to connector 147 of the dialysate outlet line 102. In this way a “bypass” may optionally be realized.

In the example of FIG. 5b, the two retainers 141 and 142, after they have passed each other, continue their path together with the connectors 145 and 147 received therein, in the direction of the arrow, respectively.

FIG. 5c shows the holder 500 according to the present disclosure, shown in FIG. 5a, in a further step of the method according to the present disclosure.

The first retainer 141 with the connector 145 of the dialysis liquid inlet line 104 received therein and the second retainer 142 with the connector 147 of the dialysate outlet line 102 received therein have each reached one of their target positions namely in the respective initial position of the other of the two retainers 141, 142 shown in FIG. 5a.

In the example in FIG. 5c, connector 145 of the dialysis liquid inlet line 104 may be connected in this target position with the second connector 147′ of the blood filter 303 (see for example FIG. 4), which is positioned relatively further down. The connector 147 of the dialysate outlet line 102 may be connected in the target position to the first connector 145′ of the blood filter 303 positioned relatively further up.

This may again be done, for example, by moving the connecting section 170 (not shown in FIG. 5c), and the blood filter 303 may be filled, flushed and/or primed from bottom to top by or with fluids from the dialysis liquid inlet line 104 connected to the connector 145.

After the connectors 145, 147, respectively received in the first retainer 141 and the second retainer 142 have been separated again from the blood filter 303, the first retainer 141 and the second retainer 142 may be moved back to their respective initial position in an analogous manner, for example, in order to connect them again, for the purpose of a blood treatment, to both connectors 145′, 147′ of the dialysis fluid chamber 303a of the blood filter 303.

FIG. 6 shows a simplified representation of an exemplary course of the method according to the present disclosure in the form of the method described above as being alternative with steps S1 to S7. In this, the reference numerals of the description of FIGS. 1 to 5c are used to describe said method.

In the context of this embodiment of the method according to the present disclosure, method step S1 is or comprises the step of providing a blood treatment apparatus 100 according to the present disclosure with a holder 500 according to the present disclosure. The holder 500 is preferably arranged at a distance from the blood filter 303 using the connecting section 170.

The connector 145 of the dialysis liquid inlet line 104 is already inserted in the first retainer 141, the connector 147 of the dialysate outlet line 102 is inserted in the second retainer 142. If a blood treatment apparatus 100 prepared in this way is already provided, step S1 is of course omitted.

In step S2, at least one of the two retainers, the first retainer 141 and/or the second retainer 142, with the connectors 145 or 147 received therein, is moved within the holder 500 such that it is in its respective target position when the movement process is completed. The target position of the respective moved retainer 141 or 142 differs from its initial position.

Method step S3 represents the movement of the holder 500 towards the blood filter 303, for example by shifting the connecting section 170. It is or further encompasses connecting the connector 145 of the dialysis liquid inlet line 104 to the second connector 147′ of the blood filter 303 and connecting the connector 147 of the dialysate outlet line 102 to the first connector 145′ of the blood filter 303.

S4 represents or encompasses a filling, rinsing and/or priming process. In this, the blood filter 303 is filled, rinsed and/or primed from the bottom to the top or dialysis liquid or another fluid from the dialysis liquid inlet line 104 flows through it or perfuses it.

Method step S5 represents the disconnection of connector 145 of the dialysis liquid inlet line 104 from the second connector 147′ of the blood filter 303 and the disconnection of connector 147 of the dialysate outlet line 102 from the first connector 145′ of the blood filter 303. The separation process may be performed, for example, by moving the connecting section 170, and thus the holder 500, away from the blood filter 303.

Step S6 represents, analogous to method step S3, the movement of the first retainer 141 and the second retainer 142 with the connectors 145 and 147 received therein, within the holder 500 or by it. The movement takes place in such a way that after the movement process has ended, the first retainer 141 is in a position in which the connector 145 of the dialysis liquid inlet line 104 received therein can be assigned to the first connector 145′ of the blood filter 303 and that the second retainer 142 is in a position in which the connector 147 of the dialysate outlet line 102 received therein can be assigned to the second connector 147′of the blood filter 303.

Method step S7 represents the movement of the holder 500 towards the blood filter 303, for example by moving the connecting section 170. Subsequently, connecting the connector 145 of the dialysis liquid inlet line 104 to the first connector 145′ of the blood filter 303 located further up, and connecting the connector 147 of the dialysate outlet line 102 to the second connector 147′ of the blood filter 303 located further down is carried out.

Subsequent to the method according to the present disclosure, a patient's blood may be treated by the blood treatment apparatus 100 according to the present disclosure. In this, the dialysis liquid may flow through the dialysis liquid chamber 303aof the blood filter 303—as is preferred—from top to bottom.

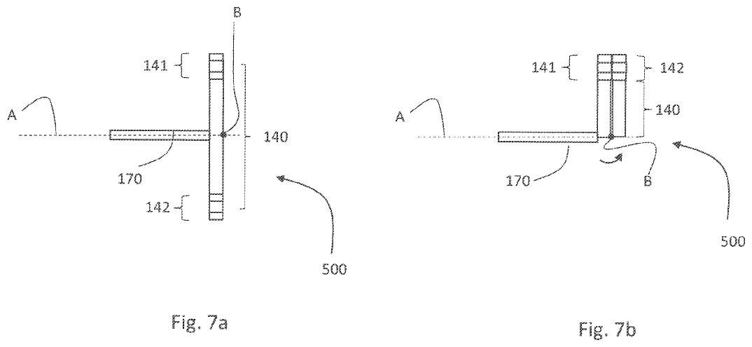

FIG. 7a shows a further embodiment of the holder 500 according to the present disclosure in in a simplified representation.

In this embodiment of the holder 500, the lower half of the arm 140 with the second retainer 142 may be folded about an axis of rotation B which is perpendicular to the plane of the drawing in FIG. 7a.

FIG. 7b shows in a simplified representation the holder 500 according to the present disclosure as shown in FIG. 7a in a folded position of the arm 140.

The folding of the first retainer 141 and the second retainer 142 may advantageously enable to bypass the dialysis liquid chamber 303a of the blood filter 303 for the fluid from the dialysis liquid inlet line 104.

One of the openings in the dialysis liquid inlet line 104 or in the dialysate outlet line 102 may be made larger than the other opening Particularly in this way, an insertion into one another may be achieved such that a fluidic connection between the dialysis liquid inlet line 104 and the dialysate outlet line 102 is achieved or established.

However, since both connectors 145, 147 at the retainers 141, 142 must be connectable to the same connectors 145′, 147′ of the blood filter 303, the connectors 145, 147 at the retainers 141, 142 may have, in addition to the connection geometry suitable for connecting to the blood filter 303, a second connection geometry which is suitable for connecting the connectors 145, 147 to each other. In this, “connecting” has the meaning that a fluidic connection of the lumens realized in the connectors 145, 147 is established.

In addition, a connecting tube or connecting hose may be formed on or in the blood treatment apparatus 100 or dialysis machine. The first retainer 141 and second retainer 142 may approach this connecting hose in such a way that they are connected to the connecting hose. Thereby, the connection is made directly via the first retainer 141 with the connecting hose on one side and the connection is made directly via the second retainer 142 with the other side of the connecting hose. Alternatively, the connection may be made using the connectors received in the first retainer 141 and second retainer 142. The connecting hose may be reached by turning and/or shifting the arm and/or turning and/or shifting the first and second retainer 141, 142.

To minimize the risk of possible contamination of the first connector 145′ of the dialysis liquid chamber 303a of the blood filter 303 by its contact with the second connector 147 of the dialysate outlet line 102, it may be advantageous to place, during use, covers 185 between, e.g., connectors 145′ and 147 which cover the whole surface, especially the hard plastic parts of the connectors 147 or 145′, or a part thereof.

Covers 185 are preferably single-use articles.

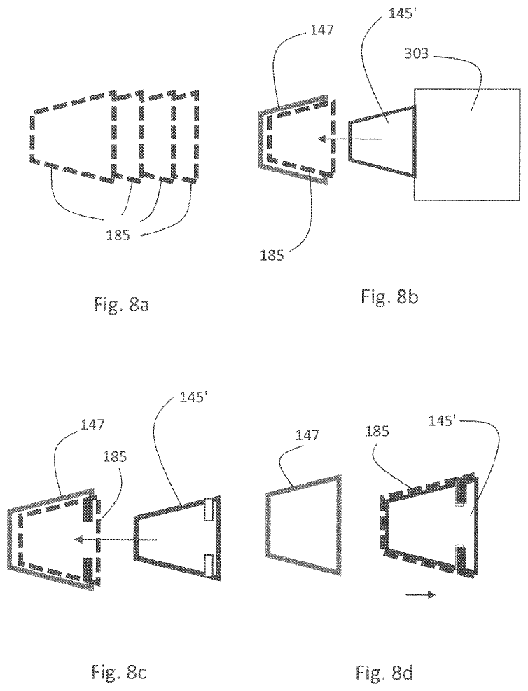

FIG. 8a shows a first design of the cover 185 and further, for example, how a plurality of them, may be stored e.g. on or at the blood treatment apparatus 100.

The design of the cover 185 in the form of, e.g., a hollow truncated cone, allows several covers 185 to be pushed into each other. This advantageously enables a very space-saving storage of several covers 185, for example 30 pieces, 50 pieces or more, in or on the blood treatment apparatus 100.

Covers 185 may, for example, comprises or consist of low molecular weight polypropylene, polyvinyl chloride (PVC) or silicone.

FIG. 8b shows the arrangement of the cover 185 shown in FIG. 8a, during use.

A cover 185 is placed, e.g., between the second connector 147 of the dialysate outlet line 102 (not shown in FIG. 8b) and the first connector 145′ of the blood filter 303.

FIG. 8c shows a further design of a cover 185. It is shown here during use.

In the example in FIG. 8c, a device for latching, for example in the form of noses, is outlined at one end of the cover 185, preferably at its wider end (here of a truncated cone). The device for latching the cover 185 corresponds to matches on the first connector 145′ of the blood filter 303. Grooves may be provided there, for example, in which the noses of the cover 185 may engage. Alternatively, the cover completely encloses the first connector 145′ and latches with it even without grooves.

FIG. 8d shows the design of the cover 185 of FIG. 8c after disconnecting the second connector 147 of the dialysate outlet line 102 from the first connector 145′ of the blood filter 303.

When disconnecting, the cover 185, which is particularly releasably connected to the first connector 145′ of the blood filter 303, is removed together with the latter from the second connector 147.

Alternatively or in addition to the latching device, other releasable connections may be provided, for example snap-on or clip-on devices but also different surface textures of the inside and outside of the cover 185, different materials and the like.

In some embodiments, such covers may also be provided as hygiene measures on different or other connector pairs than those mentioned herein, in particular when connecting the second connector 147′ of the dialysis liquid chamber 303a of the blood filter 303 to the connector 145 of the dialysis liquid inlet line 104.

### LIST OF REFERENCE NUMERALS

25 addition site for Heparin (optional)
29 venous blood chamber (optional)
31 de-aeration device
100 blood treatment apparatus
101 blood pump
102 dialysate outlet line, effluent supply line
104 dialysis liquid inlet line
111 pump for substituate
121 pump for dialysis liquid
131 pump for dialysate or effluent in effluent inlet line
140 arm
141 first retainer
142 second retainer
145 connector of the dialysis liquid inlet line
145′ first connector of the blood filter
147 connector of the dialysate outlet line
147′ second connector of the blood filter
149 guide or rail
150 control device or closed-loop control device
170 connecting section
171 rotation section
185 cover
200 source with dialysis liquid
201 source with substituate
300 extracorporeal blood circuit
301 first line (arterial line section)
302 (first) hose clamp
303 blood filter or dialyzer
303a dialysis liquid chamber
303b blood chamber
303c semi-permeable membrane
305 second line (venous line section)
306 (second) hose clamp
400 effluent bag
403 effluent outlet line
500 holder
600 basin
A rotation axis of the arm
B folding axis of the arm
H2 bag heating with bag (dialysis liquid)
H1 bag heating with bag (substituate)
PS1, PS2 arterial pressure sensor (optional)
PS3 pressure sensor (optional)
PS4 pressure sensor for measuring the filtrate pressure

1-15. (canceled)

16. A holder for a blood treatment apparatus, the holder comprising:
an arm, wherein the arm comprises:
a first retainer for receiving and/or holding a dialysis liquid inlet line; and
a second retainer for receiving and/or holding a dialysate outlet line.

17. The holder according to claim 16, wherein the holder further comprises a displacement mechanism and is arranged such that a position of the first retainer and a position of the second retainer are exchangeable or substantially exchangeable by the displacement mechanism.

18. The holder according to claim 16, wherein the arm is rotatable about, or relative to, a section of the holder.

19. The holder according to claim 18, wherein the arm is rotatable about the section of the holder substantially by 180 degrees.

20. The holder according to claim 16, wherein the first retainer and/or the second retainer are arranged in a displaceable manner along the arm.

21. The holder according to claim 20, wherein the first retainer and/or the second retainer are displaceable along the arm by a guide.

22. The holder according to claim 20, wherein the holder comprises an actuator for displacing the first retainer and/or the second retainer along the arm.

23. The holder according to claim 16, wherein the holder comprises a connecting section designed to connect the holder to a rotation section of the blood treatment apparatus generating a rotation-proof connection between the connecting section and the rotation section.

24. The holder according to claim 16, wherein the arm is variable in length.

25. The holder according to clam 24, wherein the arm is foldable or telescopic.

26. The holder according to claim 16, wherein the first retainer and the second retainer are arranged to be pivotable relative to each other, wherein a section of the holder is designed such that when the first and/or the second retainer is pivoted, a fluidic connection may be formed between the dialysis liquid inlet line and the dialysate outlet line, or
wherein the arm is designed to be foldable such that the first retainer and the second retainer are movable towards each other, wherein the dialysis liquid inlet line and the dialysate outlet line are connectable such that a fluidic connection may be formed between the dialysis liquid inlet line and the dialysate outlet line.

27. The holder according to claim 26, wherein the first retainer and the second retainer are arranged to be pivotable relative to each other and the section of the holder is the arm.

28. The holder according to claim 26, wherein the arm is designed to be foldable and the dialysis liquid inlet line and the dialysate outlet line are pluggable to each other via a connecting piece.

29. A blood treatment apparatus comprising a holder according to claim 16, wherein the blood treatment apparatus further comprises a dialysis liquid inlet line connected to or comprising a connector and a dialysate outlet line connected to or comprising a connector, and
wherein the connector associated with the dialysis liquid inlet line is part of the first retainer, received therein, held thereby and/or designed as the first retainer, and
wherein the connector associated with the dialysate outlet line, is part of the second retainer, received therein, held thereby and/or designed as the second retainer.

30. The blood treatment apparatus according to claim 29, further comprising an actuator for placing and/or removing a cover on or from the connector associated with the dialysis liquid inlet line and/or on or from the connector associated with the dialysate outlet line.

31. The blood treatment apparatus according to claim 29, wherein the connector associated with the dialysis liquid inlet line and/or the connector associated with the dialysate outlet line comprises a cover releasably mounted thereon.

32. The blood treatment apparatus according to claim 29, wherein the connector associated with the dialysis liquid inlet line, the connector associated with the dialysate outlet line, and/or the cover, or respectively a section thereof, comprises a tapering shape.

33. The blood treatment apparatus according to claim 29, further comprising a blood filter having a first connector and a second connector for connecting the blood filter to fluid lines, wherein the first connector of the blood filter, the second connector of the blood filter, the connector associated with the dialysis liquid inlet line, and/or the connector associated with the dialysate outlet line comprises a drip protection.

34. The blood treatment apparatus according to claim 29, comprising a holding device for holding or receiving a plurality of covers.

35. A method for setting up a blood treatment apparatus for carrying out a blood treatment session using the blood treatment apparatus, the method comprising:
connecting a second connector of a blood filter to a connector associated with a dialysis liquid inlet line and connecting a first connector of the blood filter to a connector associated with a dialysate outlet line, such that when liquid is supplied via the dialysis liquid inlet line the liquid flows against gravity within the blood filter in the direction towards the dialysate outlet line;
filling the blood filter at least partially with liquid via the dialysis liquid inlet line;
moving the connector associated with the dialysate outlet line and the connector associated with the dialysis liquid inlet line by a holder for holding or receiving both the dialysate outlet line and the dialysis liquid inlet line;
connecting the first connector of the blood filter to the connector associated with the dialysis liquid inlet line and connecting the second connector of the blood filter to the connector associated with the dialysate outlet line, such that when the liquid is supplied via the dialysis liquid inlet line the liquid flows with gravity within the blood filter in the direction towards the dialysate outlet line
